(19) United States
(12) Patent Application Publication
(10) Pub. No.: US 2015/0148669 A1
(43) Pub. Date: May 28, 2015

(54) MEDICAL METAL MATERIAL FOR IN VIVO INSERTION, COMPRISING IN VIVO MOVEMENT-PREVENTING MEANS

(71) Applicant: SNU R&DB FOUNDATION, Seou (KR)

(72) Inventors: Sung-Joon YE, Yongin-si (KR); Young Bin Choy, Seongnam-si (KR); Hyun Seok Lee, Seoul (KR); Sung Yoon Choi, Seongnam-si (KR); Won Seok Lee, Seoul (KR)

(21) Appl. No.: 14/579,662

(22) Filed: Dec. 22, 2014

Related U.S. Application Data
(63) Continuation-in-part of application No. PCT/KR2013/005510, filed on Jun. 21, 2013.

(30) Foreign Application Priority Data
Jun. 22, 2012 (KR) 10-2012-0067428
Jun. 21, 2013 (KR) 10-2013-0071641

(51) Int. Cl. A61N 5/10; A61B 17/08; A61L 31/02; A61L 31/14; A61L 31/10; A61B 19/00; A61L 31/18

(52) U.S. Cl. CPC A61N 5/1001; A61B 19/54; A61B 17/083; A61L 31/18; A61L 31/148; A61L 31/10; A61L 31/022; A61N 2005/1092; A61N 2005/1024; A61B 2019/5491
USPC 600/424; 600/8; 606/151

(57) ABSTRACT

The present invention relates to an implantable medical appliance with means of migration prevention, which is either coated with a biocompatible polymer or coated with a biocompatible adhesive, or injected with a biocompatible adhesive after the insertion of the implantable medical appliance into a living body, or equipped with a foldable anchor. The implantable medical appliance of the present invention characterized by the means equipped on the surface of the same to prevent intratissue migration can be effectively used for such implantable medical appliance as a sealed source used for brachytherapy, a fiducial marker used for increasing accuracy of IGRT, a clip for surgical operation, and a transponder for the generation of radio frequency, etc, since the migration of the medical appliance is prevented after the insertion.

### CROSS-REFERENCE TO RELATED APPLICATIONS

This application is a continuation-in-part of PCT/KR2013/005510, filed Jun. 21, 2013, which in turn claims the benefit of Korean Patent Application Nos. 10-2012-0067428 and 10-2013-0071641, filed Jun. 22, 2012 and Jun. 21, 2013, respectively, the entire disclosure of each of which is incorporated herein by reference.

### BACKGROUND OF THE INVENTION

1. Field of the Invention

The present invention relates to an implantable medical appliance with means of migration prevention, which is either coated with a biocompatible polymer or coated with a biocompatible adhesive, or injected with a biocompatible adhesive after the insertion of the implantable medical appliance into a living body, or equipped with a foldable anchor.

2. Description of the Related Art

There are two ways to treat cancers such as breast cancer and prostate cancer with radiation: which are image guided radiation therapy (IGRT) that is to irradiate a tumor locally from outside the body by using a radiation therapy equipment like linear accelerator and brachytherapy that is to insert a sealed source such as 1-125, Ir-192, Cs-137, and Pd-103 directly into the tumor tissue.

The conventional radiotherapy uses the image of a patient only to set a treatment plan before the treatment and once the treatment begins the image has not been used. After setting up a patient lying down for radiotherapy, laser is arranged to hit the marked area on the surface of a patient's body, indicating local irradiation on a specific target area. In this case, there might be an error from a few mm to over 1 cm, caused in the course of patient setting for irradiation, compared with the original plan for the treatment.

To solve the above problem, attempts have been made to tract the location and morphological changes of a target tumor tissue before and in the middle of the treatment. As a result, image guided radiation therapy (IGRT) has been developed.

IGRT uses a fiducial marker to narrow down to an exact treatment site. This marker is an artificial one that is inserted in a human body by an operation, which is then fixed in a target area or a neighboring area so as to provide information on the clear and exact location of a target for scanning for visualization using a visualization technique such as CT and MRI.

The said fiducial marker is used in the form of wires or beads composed of such metals that have a high radiopacity, for example gold or tantalum. However, the inserted metal moves slowly in the tissue over the time after the insertion, so that the information sent by the metal might not provide the accurate treatment site. In particular, when the fiducial marker is inserted in the prostatic tissue, it can be released in urine and out through the urethra over the time, suggesting that there is a problem in setting up the exact treatment site.

Brachytherapy is one of the radiotherapies to treat a tumor by implanting a radio-isotope seed directly into a treatment site.

The radio-isotope seed used herein is usually sealed in the form of a small rod, whose shape and size are similar to those of the fiducial marker.

The fiducial marker and the sealed source used for IGRT and Brachytherapy are in the shape of a rod of 3.0-5.0 mm in length and of 0.5-1.0 mm in diameter.

When the fiducial marker and the sealed source are used for radiotherapy by a health care professional, there is a problem of intratissue migration of these materials over the time, since these are small metals.

The present inventors tried to solve the above problem and at last completed this invention by proving that when an implantable medical appliance is coated with a biocompatible polymer having a high absorptance or coated with a biocompatible adhesive on the surface thereof, or a biocompatible adhesive is injected after the insertion thereof, or the implantable medical appliance is equipped with a foldable anchor on the surface thereof, the metal dose not migrate even a while after the insertion.

### SUMMARY OF THE INVENTION

It is an object of the present invention to provide an implantable medical appliance characterized by the means equipped on the surface of the same to prevent intratissue migration.

To achieve the above object, the present invention provides an implantable medical appliance characterized by the means equipped on the surface of the same to prevent intratissue migration.

Herein, the means is a biocompatible polymer coated at least on a part of the surface of the implantable medical appliance, a biocompatible adhesive coated at least on a part of the surface of the implantable medical appliance, or injected after the insertion of the implantable medical appliance, or a foldable anchor equipped on the surface of the implantable medical appliance.

### Advantageous Effect

The implantable medical appliance of the present invention characterized by the means equipped on the surface of the same to prevent intratissue migration can be effectively used for such implantable medical appliance as a sealed source used for brachytherapy, a fiducial marker used for increasing the accuracy of IGRT, a clip for surgical operation, and a transponder for the generation of radio frequency, etc, since the migration of the medical appliance is prevented after the insertion.

### BRIEF DESCRIPTION OF THE DRAWINGS

The application of the preferred embodiments of the present invention is best understood with reference to the accompanying drawings, wherein:

FIG. 1 is a set of images;
(a): the image of the sealed source or the fiducial marker coated with a biocompatible polymer at least on a part of the surface;
(b): the image showing the in vivo migration of the conventional sealed source or the fiducial marker which are not equipped with means of migration prevention; and
(c): the image illustrating that after the sealed source or the fiducial marker coated with a biocompatible polymer at least on a part of the surface are inserted in a living body, they absorb body fluid so as to be enlarged in their volume that makes them stuck in the surrounding tissue.

FIG. 2 is a diagram illustrating what would happen in vivo when the sealed source not coated with polydopamine prepared in Comparative Example 1 and when the sealed source coated with polydopamine prepared in Example 5 (left: Comparative Example 1, right: Example 5).

FIG. 3 is a set of images;
(a): the image of the sealed source or the fiducial marker equipped with a foldable anchor on the surface prepared according to a preferred embodiment of the present invention, and
(b): the image illustrating that when the sealed source or the fiducial marker equipped with a foldable anchor is inserted in a living body, the migration of the same is prevented by anchoring in the tissue.

FIG. 4 is a diagram showing the image of the sealed source for brachytherapy coated with polydopamine prepared in Example 5.

FIG. 5 is a set of photographs illustrating the image of the sealed source for brachytherapy coated with polydopamine prepared in Example 5, taken by SEM ((a): Comparative Example 1, (b): Example 5).

FIG. 6 is a set of graphs illustrating the results of X-ray photoelectron spectroscopy (XPS) with the sealed source for brachytherapy coated with polydopamine prepared in Example 5 ((a): Comparative Example 1, (b): Example 5).

FIG. 7 is a schematic diagram illustrating the measurement of the adhesive power of the sealed source for brachytherapy coated with polydopamine prepared in Example 5 onto the living tissue.

FIG. 8 is a graph illustrating the adhesive power of the sealed source for brachytherapy coated with polydopamine prepared in Example 5 onto the living tissue.

FIG. 9 is a schematic diagram illustrating the device designed by the present inventors to measure accurately the migration of the sealed source inserted in the living tissue in Experimental Example 3.

FIG. 10 is a set of images illustrating the migration of the sealed source for brachytherapy not coated with polydopamine prepared in Comparative Example in the living tissue. These images are the CT images of XY plane, XZ plane, and YZ plane before and after the forced movement.

FIG. 11 is a set of images illustrating the migration of the sealed source for brachytherapy coated with polydopamine prepared in Example 5 in the living tissue. These images are the CT images of XY plane, XZ plane, and YZ plane before and after the forced movement.

### DESCRIPTION OF THE PREFERRED EMBODIMENTS

Hereinafter, the present invention is described in detail.

The present invention provides an implantable medical appliance characterized by the means equipped on the surface of the same to prevent intratissue migration.

The surface of the implantable medical appliance of the present invention is made of a metal material, but not always limited thereto.

The said metal material is selected from the group consisting of titanium, stainless steel, iron, gold, silver, platinum, iridium, nickel, aluminium, tantalum, cobalt, chrome, and copper or an alloy composed of at least one of those metals selected from the same.

In the implantable medical appliance of the present invention, the medical appliance can be the sealed source, the fiducial marker, the clip for surgical operation, and the transponder for the generation of radio frequency, and can further be any implantable medical appliance.

The sealed source herein is exemplified by I-125, Pd-103, Ir-192, Au-198, Yb-169, Cs-131, Cs-137, and Co-60, etc, but not always limited thereto and any seed that is suitable for brachytherapy to treat cancer can be used without limitation.

The said fiducial marker can be any radiopaque material.

There are three different ways to prevent the migration of the implantable medical appliance of the present invention in the living tissue. Hereinafter, these ways are described in detail.

The first means of the present invention is the biocompatible polymer coated at least on a part of the surface of the implantable medical appliance. This biocompatible polymer is increased in its volume by absorbing body fluid in vivo.

The mechanism of preventing in vivo migration of the implantable medical appliance in which the means described above (coating with the biocompatible polymer) is applied on the surface as shown in FIG. 1.

As shown in FIG. 1, when the implantable medical appliance coated with the biocompatible polymer at least on a part of the surface is inserted into a living body, the polymer absorbs body fluid to be enlarged in its volume, so that it becomes stuck in the surrounding tissues owing to the increased volume that makes it hard to move in the tissue.

The biocompatible polymer enlarged in its volume by absorbing body fluid herein is exemplified by hydrogel such as chitosan, starch, guargum, gelatin, and collagen; polylactide (PLA), polyglycolide (PGA) or their copolymer poly(lactic-co-glycolic acid) (PLGA) having the porous structure to increase body fluid absorptance; polyester, polyorthoester, polyanhydride, polyamino acid, polyhydroxybutyric acid, polycaprolactone, polyalkylcarbonate, and ethyl cellulose, but not always limited thereto.

More preferably, the biocompatible polymer herein can be selected from the group consisting of those showing especially high volume increase by absorbing body fluid, such as chitosan, starch, guargum, gelatin, and collagen.

Further, considering the required duration of radiotherapy is approximately 60 days, the biocompatible polymer herein is supposed to start being degraded at least 60 days after the insertion into the living tissue, which favors the prevention of migration of the implantable medical appliance until the end of radiotherapy. To confirm the therapeutic effect of radiotherapy, CT or X-ray is re-taken 1-2 years later. Therefore, it is more preferred for the implantable medical appliance to start being degraded 1-2 years after the insertion into the living tissue.

The second means of migration prevention of the invention is the biocompatible adhesive coated at least on a part of the surface of the implantable medical appliance. This biocompatible adhesive can be coated on the medical appliance before implantation or be injected thereto after implantation by using insertion supporting appliance (such as, endoscope, applicator, catheter, etc). This biocompatible adhesive is not limited as long as it has excellent adhesiveness on both the medical appliance and the living tissue.

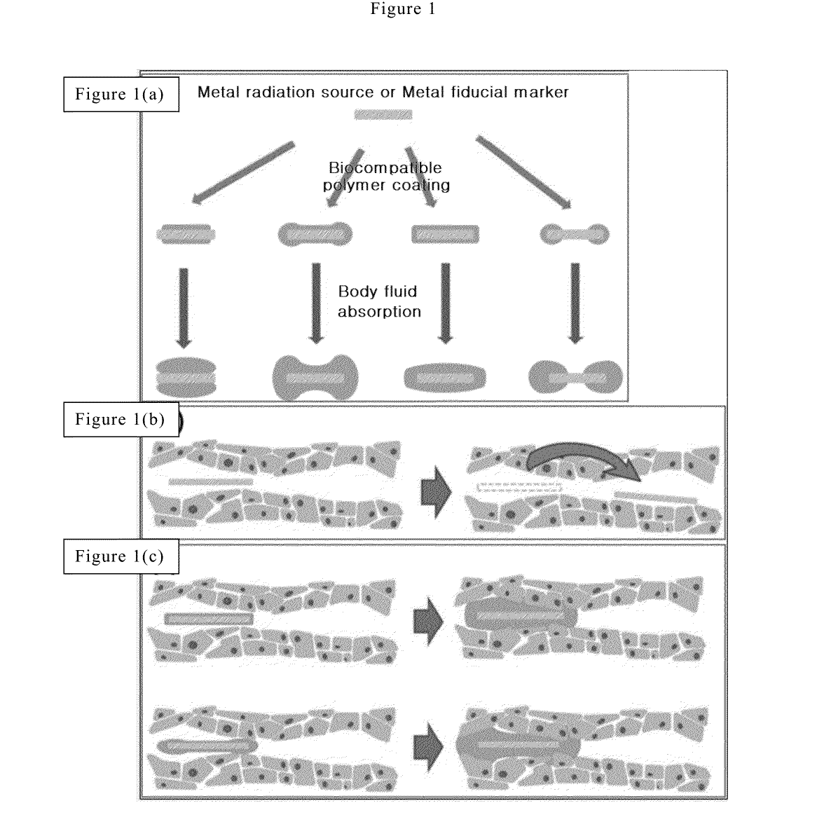

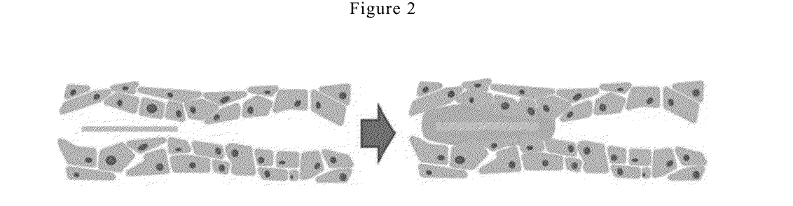

The mechanism of preventing in vivo migration of the implantable medical appliance by the means described above (coating with the biocompatible adhesive) is as shown in FIG. 2.

As shown in FIG. 2, the implantable medical appliance coated with the biocompatible adhesive at least a part of it is either coated before in vivo implantation or injected after implantation by using insertion supporting appliance (such as, endoscope, applicator, catheter, etc), by which the implantable medical appliance is surrounded by the neighboring tissues to prevent intratissue migration.

The biocompatible adhesive is exemplified by polydopamine, cyanoacrylate, fibrin glue, protein glue, polyurethane, and PEG containing sealant, etc, but not always limited thereto.

Another example of the biocompatible adhesive is Az-chitosan (Azidobenzoic acid modified chitosan) whose adhesiveness is generated by reacting a liquid or solution phase polymer with external stimulus such as UV irradiation or pH change, but not always limited thereto.

Further, considering the required duration of radiotherapy is approximately 60 days, the biocompatible polymer herein is supposed to start being degraded at least 60 days after the insertion into the living tissue, which favors the prevention of migration of the implantable medical appliance until the end of radiotherapy. To confirm the therapeutic effect of radiotherapy, CT or X-ray is re-taken 1-2 years later. Therefore, it is more preferred for the implantable medical appliance to start being degraded 1-2 years after the insertion into the living tissue.

The third means of migration prevention of the invention is the foldable anchor attached on the surface of the implantable medical appliance. This anchor is folded during the implantation but it is unfolded in a target location after the implantation in order to be successfully anchored in surrounding tissues.

The mechanism of preventing in vivo migration of the implantable medical appliance by the means described above (the foldable anchor) is as shown in FIG. 3.

As shown in FIG. 3, the foldable anchor equipped in the implantable medical appliance stays folded while it is carried in the insertion supporting appliance (such as, endoscope, applicator, catheter, etc) but once it reaches a target area after the implantation, the anchor structure is unfolded to be anchored in the surrounding tissues, resulting in the prevention of migration of the implantable medical appliance.

As explained hereinbefore, the implantable medical appliance of the present invention characterized by the means equipped on the surface of the same to prevent intratissue migration can be effectively used for such implantable medical appliance as a sealed source used for brachytherapy, a fiducial marker used for increasing accuracy of IGRT, a clip for surgical operation, and a transponder for the generation of radio frequency, etc, since the migration of the medical appliance is prevented after the insertion.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1a

### Preparation of the Sealed Source for Brachytherapy Coated with the Biocompatible Polymer with Excellent Absorptance 1

The sealed source sealed in titanium (radio-isotope seed: I-125, diameter: 0.5-1 mm, length: 5-10 mm) was used, and chitosan was used as a biocompatible polymer. The sealed source was coated with the polymer by using the standard wire coating method. Then, the coated sealed source was cut into 5-10 mm long fragments, resulting in the preparation of the sealed source coated with chitosan.

### Example 1b

### Preparation of the Sealed Source for Brachytherapy Coated with the Biocompatible Polymer with Excellent Absorptance 2

The sealed source coated with starch was prepared by the same manner as described in Example 1a except starch was used as the biocompatible polymer instead of chitosan.

### Example 1c

### Preparation of the Sealed Source for Brachytherapy Coated with the Biocompatible Polymer with Excellent Absorptance 3

The sealed source coated with guargum was prepared by the same manner as described in Example 1a except guargum was used as the biocompatible polymer instead of chitosan.

### Example 1d

### Preparation of the Sealed Source for Brachytherapy Coated with the Biocompatible Polymer with Excellent Absorptance 4

The sealed source coated with gelatin was prepared by the same manner as described in Example 1a except gelatin was used as the biocompatible polymer instead of chitosan.

### Example 1e

### Preparation of the Sealed Source for Brachytherapy Coated with the Biocompatible Polymer with Excellent Absorptance 5

The sealed source coated with collagen was prepared by the same manner as described in Example 1a except collagen was used as the biocompatible polymer instead of chitosan.

### Example 1f

### Preparation of the Sealed Source for Brachytherapy Coated with the Biocompatible Polymer with Excellent Absorptance 6

The sealed source coated with polylactide was prepared by the same manner as described in Example 1a except polylactide was used as the biocompatible polymer instead of chitosan.

### Example 1g

### Preparation of the Sealed Source for Brachytherapy Coated with the Biocompatible Polymer with Excellent Absorptance 7

The sealed source coated with polyglycolide was prepared by the same manner as described in Example 1a except polyglycolide was used as the biocompatible polymer instead of chitosan.

### Example 1h

### Preparation of the Sealed Source for Brachytherapy Coated with the Biocompatible Polymer with Excellent Absorptance 8

The sealed source coated with poly(lactic-co-glycolic acid) was prepared by the same manner as described in Example 1a except poly(lactic-co-glycolic acid) was used as the biocompatible polymer instead of chitosan.

### Example 1i

### Preparation of the Sealed Source for Brachytherapy Coated with the Biocompatible Polymer with Excellent Absorptance 9

The sealed source coated with polyester was prepared by the same manner as described in Example 1a except polyester was used as the biocompatible polymer instead of chitosan.

### Example 1j

### Preparation of the Sealed Source for Brachytherapy Coated with the Biocompatible Polymer with Excellent Absorptance 10

The sealed source coated with polyorthoester was prepared by the same manner as described in Example 1a except polyorthoester was used as the biocompatible polymer instead of chitosan.

### Example 1k

### Preparation of the Sealed Source for Brachytherapy Coated with the Biocompatible Polymer with Excellent Absorptance 11

The sealed source coated with polyanhydride was prepared by the same manner as described in Example 1a except polyanhydride was used as the biocompatible polymer instead of chitosan.

### Example 1l

### Preparation of the Sealed Source for Brachytherapy Coated with the Biocompatible Polymer with Excellent Absorptance 12

The sealed source coated with polyamino acid was prepared by the same manner as described in Example 1a except polyamino acid was used as the biocompatible polymer instead of chitosan.

### Example 1m

### Preparation of the Sealed Source for Brachytherapy Coated with the Biocompatible Polymer with Excellent Absorptance 13

The sealed source coated with polyhydroxybutyric acid was prepared by the same manner as described in Example 1a except polyhydroxybutyric acid was used as the biocompatible polymer instead of chitosan.

### Example 1n

### Preparation of the Sealed Source for Brachytherapy Coated with the Biocompatible Polymer with Excellent Absorptance 14

The sealed source coated with polycaprolactone was prepared by the same manner as described in Example 1a except polycaprolactone was used as the biocompatible polymer instead of chitosan.

### Example Lo

### Preparation of the Sealed Source for Brachytherapy Coated with the Biocompatible Polymer with Excellent Absorptance 15

The sealed source coated with polyalkylcarbonate was prepared by the same manner as described in Example 1a except polyalkylcarbonate was used as the biocompatible polymer instead of chitosan.

### Example 1p

### Preparation of the Sealed Source for Brachytherapy Coated with the Biocompatible Polymer with Excellent Absorptance 16

The sealed source coated with ethyl cellulose was prepared by the same manner as described in Example 1a except ethyl cellulose was used as the biocompatible polymer instead of chitosan.

### Example 2a

### Preparation of the Sealed Source for Brachytherapy Coated with the Biocompatible Polymer with Excellent Absorptance 17

The sealed source sealed in gold (radio-isotope seed: Pd-103, diameter: 0.5-1 mm, length: 5-10 mm) was used, and chitosan was used as a biocompatible polymer. The sealed source was coated with the polymer by using the standard wire coating method. Then, the coated sealed source was cut into 5-10 mm long fragments, resulting in the preparation of the sealed source coated with chitosan.

### Example 2b

### Preparation of the Sealed Source for Brachytherapy Coated with the Biocompatible Polymer with Excellent Absorptance 18

The sealed source coated with starch was prepared by the same manner as described in Example 2a except starch was used as the biocompatible polymer instead of chitosan.

### Example 2c

### Preparation of the Sealed Source for Brachytherapy Coated with the Biocompatible Polymer with Excellent Absorptance 19

The sealed source coated with guargum was prepared by the same manner as described in Example 2a except guargum was used as the biocompatible polymer instead of chitosan.

### Example 2d

### Preparation of the Sealed Source for Brachytherapy Coated with the Biocompatible Polymer with Excellent Absorptance 20

The sealed source coated with gelatin was prepared by the same manner as described in Example 2a except gelatin was used as the biocompatible polymer instead of chitosan.

### Example 2e

### Preparation of the Sealed Source for Brachytherapy Coated with the Biocompatible Polymer with Excellent Absorptance 21

The sealed source coated with collagen was prepared by the same manner as described in Example 2a except collagen was used as the biocompatible polymer instead of chitosan.

### Example 2f

### Preparation of the Sealed Source for Brachytherapy Coated with the Biocompatible Polymer with Excellent Absorptance 22

The sealed source coated with polylactide was prepared by the same manner as described in Example 2a except polylactide was used as the biocompatible polymer instead of chitosan.

### Example 2g

### Preparation of the Sealed Source for Brachytherapy Coated with the Biocompatible Polymer with Excellent Absorptance 23

The sealed source coated with polyglycolide was prepared by the same manner as described in Example 2a except polyglycolide was used as the biocompatible polymer instead of chitosan.

### Example 2h

### Preparation of the Sealed Source for Brachytherapy Coated with the Biocompatible Polymer with Excellent Absorptance 24

The sealed source coated with poly(lactic-co-glycolic acid) was prepared by the same manner as described in Example 2a except poly(lactic-co-glycolic acid) was used as the biocompatible polymer instead of chitosan.

### Example 2i

### Preparation of the Sealed Source for Brachytherapy Coated with the Biocompatible Polymer with Excellent Absorptance 25

The sealed source coated with polyester was prepared by the same manner as described in Example 2a except polyester was used as the biocompatible polymer instead of chitosan.

### Example 2j

### Preparation of the Sealed Source for Brachytherapy Coated with the Biocompatible Polymer with Excellent Absorptance 26

The sealed source coated with polyorthoester was prepared by the same manner as described in Example 2a except polyorthoester was used as the biocompatible polymer instead of chitosan.

### Example 2k

### Preparation of the Sealed Source for Brachytherapy Coated with the Biocompatible Polymer with Excellent Absorptance 27

The sealed source coated with polyanhydride was prepared by the same manner as described in Example 2a except polyanhydride was used as the biocompatible polymer instead of chitosan.

### Example 21

### Preparation of the Sealed Source for Brachytherapy Coated with the Biocompatible Polymer with Excellent Absorptance 28

The sealed source coated with polyamino acid was prepared by the same manner as described in Example 2a except polyamino acid was used as the biocompatible polymer instead of chitosan.

### Example 2m

### Preparation of the Sealed Source for Brachytherapy Coated with the Biocompatible Polymer with Excellent Absorptance 29

The sealed source coated with polyhydroxybutyric acid was prepared by the same manner as described in Example 2a except polyhydroxybutyric acid was used as the biocompatible polymer instead of chitosan.

### Example 2n

### Preparation of the Sealed Source for Brachytherapy Coated with the Biocompatible Polymer with Excellent Absorptance 30

The sealed source coated with polycaprolactone was prepared by the same manner as described in Example 2a except polycaprolactone was used as the biocompatible polymer instead of chitosan.

### Example 2o

### Preparation of the Sealed Source for Brachytherapy Coated with the Biocompatible Polymer with Excellent Absorptance 31

The sealed source coated with polyalkylcarbonate was prepared by the same manner as described in Example 2a except polyalkylcarbonate was used as the biocompatible polymer instead of chitosan.

### Example 2p

### Preparation of the Sealed Source for Brachytherapy Coated with the Biocompatible Polymer with Excellent Absorptance 32

The sealed source coated with ethyl cellulose was prepared by the same manner as described in Example 2a except ethyl cellulose was used as the biocompatible polymer instead of chitosan.

### Example 3a

### Preparation of the Sealed Source for Brachytherapy Coated with the Biocompatible Polymer with Excellent Absorptance 33

The sealed source sealed in stainless (radio-isotope seed: Ir-192, diameter: 0.5-1 mm, length: 5-10 mm) was used, and chitosan was used as a biocompatible polymer. The sealed source was coated with the polymer by using the standard wire coating method. Then, the coated sealed source was cut into 5-10 mm long fragments, resulting in the preparation of the sealed source coated with chitosan.

### Example 3b

### Preparation of the Sealed Source for Brachytherapy Coated with the Biocompatible Polymer with Excellent Absorptance 34

The sealed source coated with starch was prepared by the same manner as described in Example 3a except starch was used as the biocompatible polymer instead of chitosan.

### Example 3c

### Preparation of the Sealed Source for Brachytherapy Coated with the Biocompatible Polymer with Excellent Absorptance 35

The sealed source coated with guargum was prepared by the same manner as described in Example 3a except guargum was used as the biocompatible polymer instead of chitosan.

### Example 3d

### Preparation of the Sealed Source for Brachytherapy Coated with the Biocompatible Polymer with Excellent Absorptance 36

The sealed source coated with gelatin was prepared by the same manner as described in Example 3a except gelatin was used as the biocompatible polymer instead of chitosan.

### Example 3e

### Preparation of the Sealed Source for Brachytherapy Coated with the Biocompatible Polymer with Excellent Absorptance 37

The sealed source coated with collagen was prepared by the same manner as described in Example 3a except collagen was used as the biocompatible polymer instead of chitosan.

### Example 3f

### Preparation of the Sealed Source for Brachytherapy Coated with the Biocompatible Polymer with Excellent Absorptance 38

The sealed source coated with polylactide was prepared by the same manner as described in Example 3a except polylactide was used as the biocompatible polymer instead of chitosan.

### Example 3g

### Preparation of the sealed source for brachytherapy coated with the biocompatible polymer with excellent absorptance 39

The sealed source coated with polyglycolide was prepared by the same manner as described in Example 3a except polyglycolide was used as the biocompatible polymer instead of chitosan.

### Example 3h

### Preparation of the Sealed Source for Brachytherapy Coated with the Biocompatible Polymer with Excellent Absorptance 40

The sealed source coated with poly(lactic-co-glycolic acid) was prepared by the same manner as described in Example 3a except poly(lactic-co-glycolic acid) was used as the biocompatible polymer instead of chitosan.

### Example 3i

### Preparation of the Sealed Source for Brachytherapy Coated with the Biocompatible Polymer with Excellent Absorptance 41

The sealed source coated with polyester was prepared by the same manner as described in Example 3a except polyester was used as the biocompatible polymer instead of chitosan.

### Example 3j

### Preparation of the Sealed Source for Brachytherapy Coated with the Biocompatible Polymer with Excellent Absorptance 42

The sealed source coated with polyorthoester was prepared by the same manner as described in Example 3a except polyorthoester was used as the biocompatible polymer instead of chitosan.

### Example 3k

### Preparation of the Sealed Source for Brachytherapy Coated with the Biocompatible Polymer with Excellent Absorptance 43

The sealed source coated with polyanhydride was prepared by the same manner as described in Example 3a except polyanhydride was used as the biocompatible polymer instead of chitosan.

### Example 31

### Preparation of the Sealed Source for Brachytherapy Coated with the Biocompatible Polymer with Excellent Absorptance 44

The sealed source coated with polyamino acid was prepared by the same manner as described in Example 3a except polyamino acid was used as the biocompatible polymer instead of chitosan.

### Example 3m

### Preparation of the Sealed Source for Brachytherapy Coated with the Biocompatible Polymer with Excellent Absorptance 45

The sealed source coated with polyhydroxybutyric acid was prepared by the same manner as described in Example 3a except polyhydroxybutyric acid was used as the biocompatible polymer instead of chitosan.

### Example 3n

### Preparation of the Sealed Source for Brachytherapy Coated with the Biocompatible Polymer with Excellent Absorptance 46

The sealed source coated with polycaprolactone was prepared by the same manner as described in Example 3a except polycaprolactone was used as the biocompatible polymer instead of chitosan.

### Example 3o

### Preparation of the Sealed Source for Brachytherapy Coated with the Biocompatible Polymer with Excellent Absorptance 47

The sealed source coated with polyalkylcarbonate was prepared by the same manner as described in Example 3a except polyalkylcarbonate was used as the biocompatible polymer instead of chitosan.

### Example 3p

### Preparation of the Sealed Source for Brachytherapy Coated with the Biocompatible Polymer with Excellent Absorptance 48

The sealed source coated with ethyl cellulose was prepared by the same manner as described in Example 3a except ethyl cellulose was used as the biocompatible polymer instead of chitosan.

### Example 4a

### Preparation of the Fiducial Marker Coated with the Biocompatible Polymer with Excellent Absorptance 1

Stainless steel wire (diameter: 0.5-1 mm) was used as a fiducial marker, and chitosan was used as a biocompatible polymer. The wire was coated with the polymer by using the standard wire coating method. Then, the coated fiducial marker was cut into 5-10 mm long fragments, resulting in the preparation of the fiducial marker coated with the biocompatible polymer.

### Example 4b

### Preparation of the Fiducial Marker Coated with the Biocompatible Polymer with Excellent Absorptance 2

The fiducial marker coated with the biocompatible polymer was prepared by the same manner as described in Example 4a except starch was used as the biocompatible polymer instead of chitosan.

### Example 4c

### Preparation of the Fiducial Marker Coated with the Biocompatible Polymer with Excellent Absorptance 3

The fiducial marker coated with the biocompatible polymer was prepared by the same manner as described in Example 4a except guargum was used as the biocompatible polymer instead of chitosan.

### Example 4d

### Preparation of the Fiducial Marker Coated with the Biocompatible Polymer with Excellent Absorptance 4

The fiducial marker coated with the biocompatible polymer was prepared by the same manner as described in Example 4a except gelatin was used as the biocompatible polymer instead of chitosan.

### Example 4e

### Preparation of the Fiducial Marker Coated with the Biocompatible Polymer with Excellent Absorptance 5

The fiducial marker coated with the biocompatible polymer was prepared by the same manner as described in Example 4a except collagen was used as the biocompatible polymer instead of chitosan.

### Example 4f

### Preparation of the Fiducial Marker Coated with the Biocompatible Polymer with Excellent Absorptance 6

The fiducial marker coated with the biocompatible polymer was prepared by the same manner as described in Example 4a except polylactide was used as the biocompatible polymer instead of chitosan.

### Example 4g

### Preparation of the Fiducial Marker Coated with the Biocompatible Polymer with Excellent Absorptance 7

The fiducial marker coated with the biocompatible polymer was prepared by the same manner as described in Example 4a except polyglycolide was used as the biocompatible polymer instead of chitosan.

### Example 4h

### Preparation of the Fiducial Marker Coated with the Biocompatible Polymer with Excellent Absorptance 8

The fiducial marker coated with the biocompatible polymer was prepared by the same manner as described in Example 4a except poly(lactic-co-glycolic acid) was used as the biocompatible polymer instead of chitosan.

### Example 4i

### Preparation of the Fiducial Marker Coated with the Biocompatible Polymer with Excellent Absorptance 9

The fiducial marker coated with the biocompatible polymer was prepared by the same manner as described in Example 4a except polyester was used as the biocompatible polymer instead of chitosan.

### Example 4j

### Preparation of the Fiducial Marker Coated with the Biocompatible Polymer with Excellent Absorptance 10

The fiducial marker coated with the biocompatible polymer was prepared by the same manner as described in Example 4a except polyorthoester was used as the biocompatible polymer instead of chitosan.

### Example 4k

### Preparation of the Fiducial Marker Coated with the Biocompatible Polymer with Excellent Absorptance 11

The fiducial marker coated with the biocompatible polymer was prepared by the same manner as described in Example 4a except polyanhydride was used as the biocompatible polymer instead of chitosan.

### Example 41

### Preparation of the Fiducial Marker Coated with the Biocompatible Polymer with Excellent Absorptance 12

The fiducial marker coated with the biocompatible polymer was prepared by the same manner as described in Example 4a except polyamino acid was used as the biocompatible polymer instead of chitosan.

### Example 4m

### Preparation of the Fiducial Marker Coated with the Biocompatible Polymer with Excellent Absorptance 13

The fiducial marker coated with the biocompatible polymer was prepared by the same manner as described in Example 4a except polyhydroxybutyric acid was used as the biocompatible polymer instead of chitosan.

### Example 4n

### Preparation of the Fiducial Marker Coated with the Biocompatible Polymer with Excellent Absorptance 14

The fiducial marker coated with the biocompatible polymer was prepared by the same manner as described in Example 4a except polycaprolactone was used as the biocompatible polymer instead of chitosan.

### Example 4o

### Preparation of the Fiducial Marker Coated with the Biocompatible Polymer with Excellent Absorptance 15

The fiducial marker coated with the biocompatible polymer was prepared by the same manner as described in Example 4a except starch was used as the biocompatible polymer instead of chitosan.

### Example 4p

### Preparation of the Fiducial Marker Coated with the Biocompatible Polymer with Excellent Absorptance 16

The fiducial marker coated with the biocompatible polymer was prepared by the same manner as described in Example 4a except ethyl cellulose was used as the biocompatible polymer instead of chitosan.

### Example 5

### Preparation of the Sealed Source Coated with the Biocompatible Adhesive

Dopamine was added to tris-buffer (10 mM) at the concentration of 10 mg/ml. pH of the mixture was regulated to be 8.5. The sealed source sealed in titanium (radio-isotope seed: I-125, diameter: 0.5-1 mm, length: 5-10 mm) was soaked in the mixture for 12 hours, resulting in the preparation of the sealed source coated with polydopamine as the biocompatible adhesive.

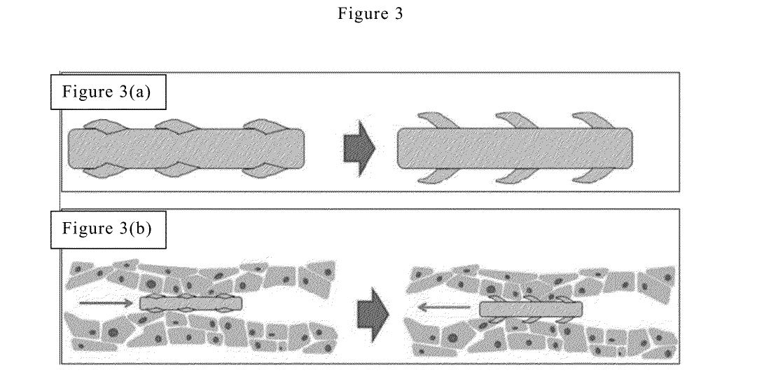

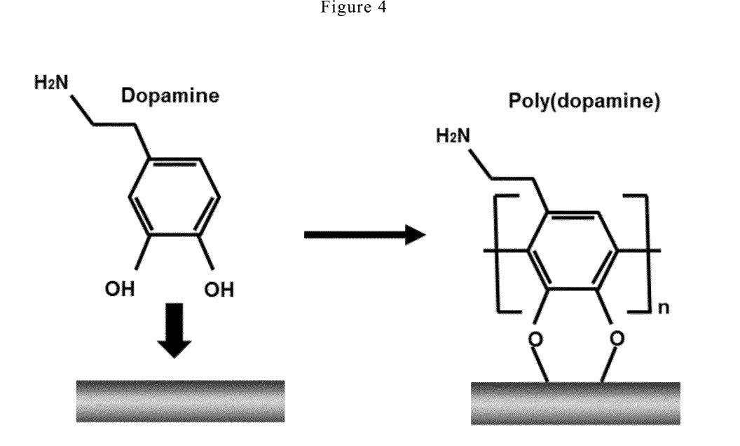

FIG. 4 shows the image of the sealed source for brachytherapy coated with polydopamine prepared in Example 5.

### Comparative Example 1

### Preparation of the Sealed Source Not-Coated with the Biocompatible Adhesive

The sealed source sealed in titanium (radio-isotope seed: I-125, diameter: 0.5-1 mm, length: 5-10 mm) used in Example 5 was prepared without polydopamine coating as the comparative example.

### Experimental Example 1

### Evaluation of the Biocompatible Adhesive Coating on the Sealed Source for Brachytherapy

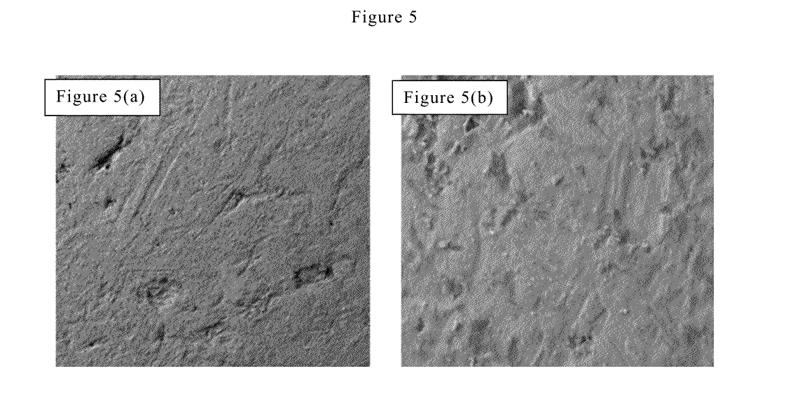

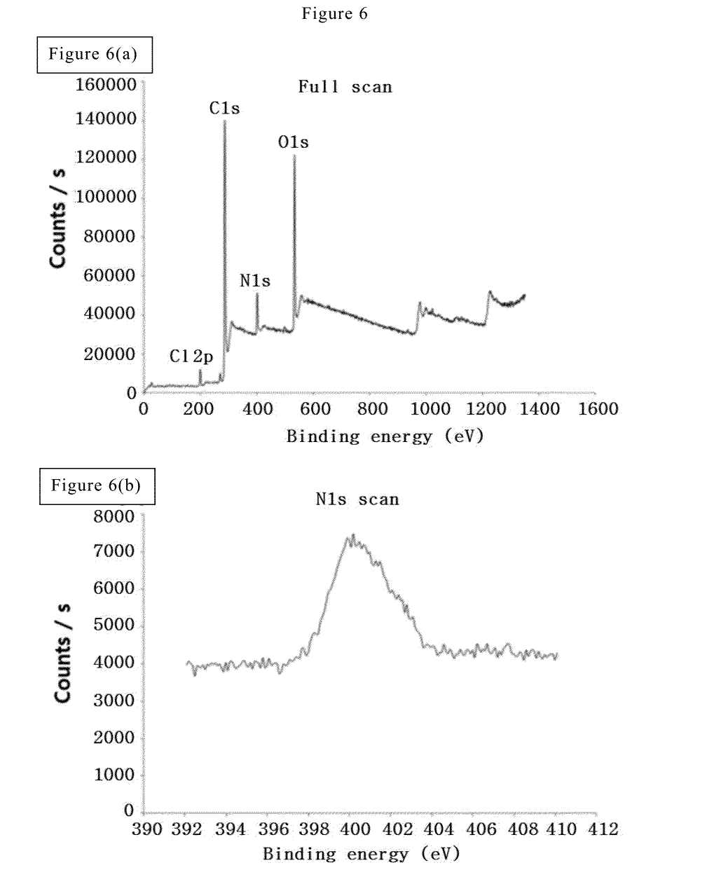

To investigate whether or not the sealed source for brachytherapy was successfully coated with the biocompatible adhesive (polydopamine) prepared in Example 5, observation under scanning electron microscope (SEM) (7410F, Jeol, Japan) and evaluation with X-ray photoelectron spectroscopy (XPS) (K-Alpha, Thermo Scientific Inc., Ltd.) were performed. The results are shown in FIG. 5 and FIG. 6.

FIG. 5 is a set of photographs illustrating the image of the sealed source for brachytherapy coated with polydopamine prepared in Example 5, taken by SEM ((a): Comparative Example 1, (b): Example 5).

FIG. 6 is a set of graphs illustrating the results of X-ray photoelectron spectroscopy (XPS) with the sealed source for brachytherapy coated with polydopamine prepared in Example 5 ((a): Comparative Example 1, (b): Example 5).

As shown in FIG. 5 and FIG. 6, it was confirmed by SEM image that the 1-125 seed sealed in titanium was successfully coated with polydopamine. Nitrogen (N), the element titanium did not contain, was detected by X-ray photoelectron spectroscopy (XPS), indicating that the surface of the sealed source was coated with polydopamine.

Therefore, the implantable medical appliance of the present invention can be effectively used for the preparation of those implantable medical appliance with means of migration prevention after in vivo implantation owing to the dopamine coated thereon to play an in vivo adhesive.

### Experimental Example 2

### Evaluation of Adhesiveness on Living Tissue

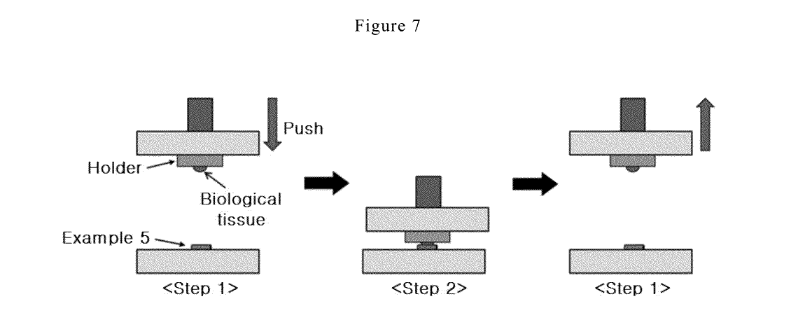

To investigate the adhesiveness of the sealed source for brachytherapy coated with the biocompatible adhesive (polydopamine) prepared in Example 5 on the living tissue, the following experiment was performed as shown in FIG. 7.

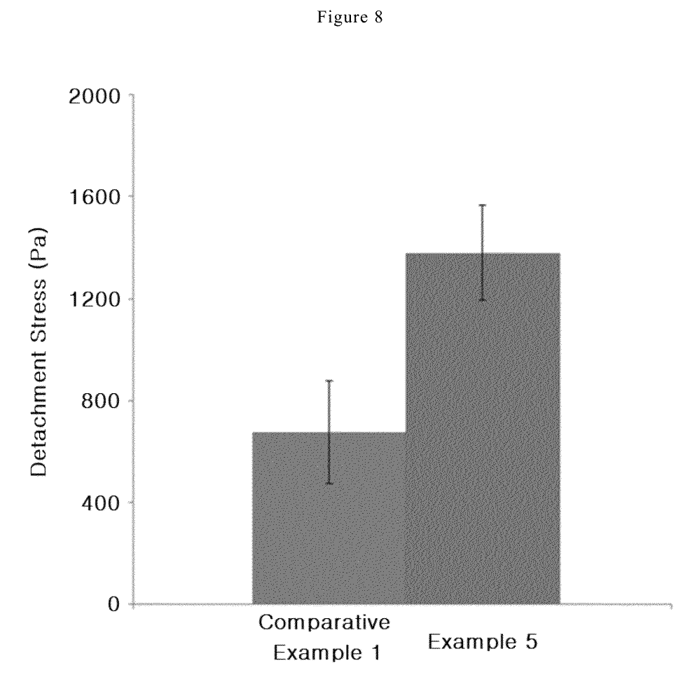

Particularly, the pig liver, as the living tissue, was placed on the top holder of UTM (universal testing machine, Instron-5543, Instron) as shown in FIG. 7. The sealed source for brachytherapy coated with polydopamine prepared in Example 5 was placed on the bottom holder, followed by measurement of detachment stress. The results are shown in Table 1 and FIG. 8.

TABLE 1

| | Detachment Stress (Pa) |
|---|---|
| Comparative Example 1 | 675 ± 202 |
| Example 5 | 1380 ± 185 |

FIG. 7 is a schematic diagram illustrating the measurement of the adhesive power of the sealed source for brachytherapy coated with polydopamine prepared in Example 5 onto the living tissue.

FIG. 8 is a graph illustrating the adhesive power of the sealed source for brachytherapy coated with polydopamine prepared in Example 5 onto the living tissue.

As shown in Table 1 and FIG. 8, the adhesiveness of the sealed source for brachytherapy coated with polydopamine prepared in Example 5 was twice as high as the adhesiveness of the sealed source for brachytherapy not-coated with polydopamine prepared in Comparative Example 1.

Therefore, it was confirmed that the implantable medical appliance of the present invention has significantly improved adhesiveness on living tissue, so that it can be effectively used for the preparation of those implantable medical appliance with means of migration prevention after in vivo implantation.

### Experimental Example 3

### Fixation Test in Living Tissue (In Vitro)

The sealed source for brachytherapy coated with the biocompatible adhesive (polydopamine) prepared in Example 5 was inserted into the living tissue. Then, the following experiment was performed to investigate the fixation of the sealed source in the living tissue under the forced movement.

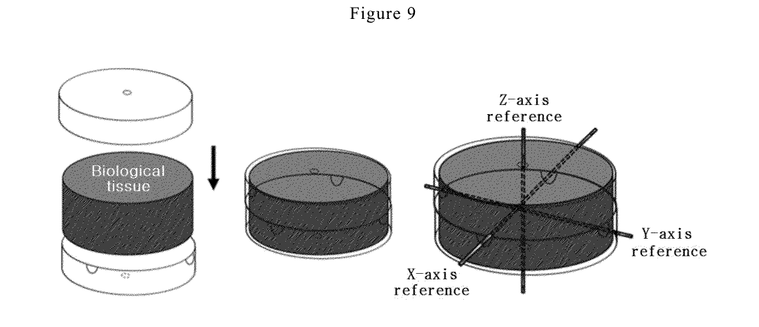

It is very hard to evaluate precisely the migration of the sealed source by deformation under the forced movement stress. So, as shown in FIG. 9, the present inventors designed “holder-reference system” first and used in this experiment. This “holder reference system” is advantageous in preventing tissue deformation and movement of reference during CT scanning, suggesting that CT scanning is performed under the same condition excluding outside variants. Therefore, only the migration of the sealed source implanted in the living tissue can be evaluated with this system.

Particularly, 2 pig liver tissues (diameter: 4 cm, height: 3 cm) were prepared as the living tissue. The sealed sources prepared in Example 5 and the other seeds prepared in Comparative Example 1 were respectively implanted, three seeds in each living tissue. The prepared living tissues were placed in the “holder-reference system” designed by the present inventors. The reference rods were inserted into the living tissue, X-axis Y-axis and Z-axis. CT was taken on XY plane, XZ plane, and YZ plane, which was the first scanning to provide the information on the location of the sealed source in the living tissue before any movement was given in the living tissue.

Then, to copy the actual blood flow of a patient, the living tissues respectively inserted with the sealed sources of Example 5 and the sealed sources of Comparative Example 1 were soaked in PBS, which was forced to move by using motion platform (VORTEX-GENIE 2, Scientific Industries, Inc.). XY plane, XZ plane, and YZ plane were scanned by CT again, which was the second scanning to provide the information on the location of the sealed source in the living tissue.

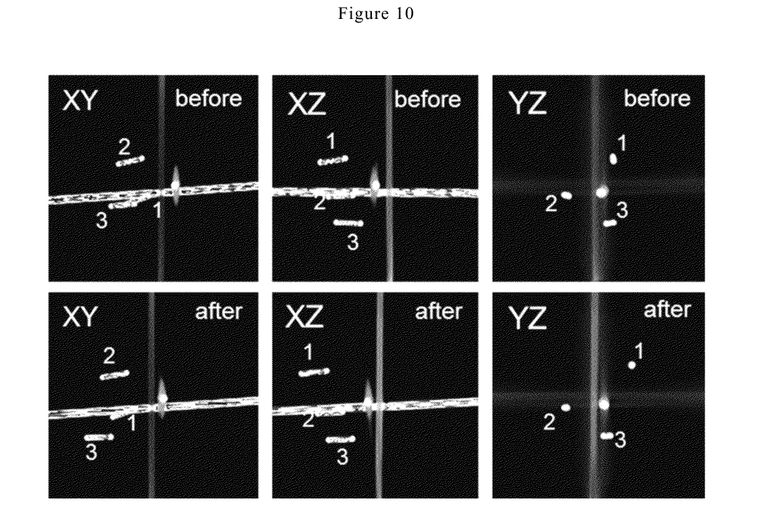

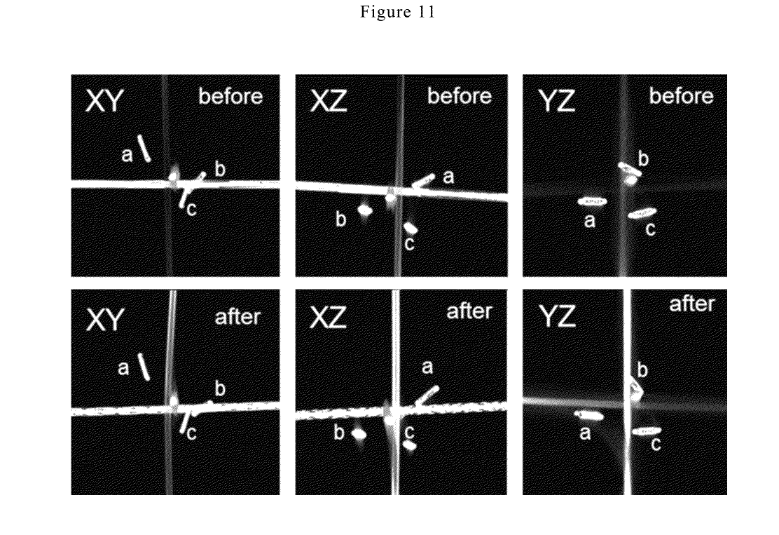

The location of the sealed source before the forced movement and the location of the sealed source after the forced movement were compared to evaluate the migration of the sealed source in the living tissue. The results are shown in Table 2 and FIGS. 10 and 11.

TABLE 2

| | | | | | Total |
|---|---|---|---|---|---|
| | | Migration distance of | | | migration |
| | | each axial direction (mm) | | | distance |
| | | X-axis | Y-axis | Z-axis | (mm) |
| Comparative | Sealed source 1 | −2.05 | −2.51 | −0.90 | 3.37 |
| Example 1 | Sealed source 2 | −0.66 | −0.49 | 0.32 | 0.88 |
| | Sealed source 3 | −1.60 | 0.39 | −0.44 | 1.70 |
| Example 5 | Sealed source a | −0.43 | −0.96 | −0.13 | 1.06 |
| | Sealed source b | −0.98 | −0.43 | −0.34 | 1.13 |
| | Sealed source c | −0.10 | 0.13 | −0.39 | 0.42 |

FIG. 10 is a set of images illustrating the migration of the sealed source not coated with polydopamine prepared in Comparative Example 1 in the living tissue. These images are the CT images of XY plane, XZ plane, and YZ plane before and after the forced movement.

FIG. 11 is a set of images illustrating the migration of the sealed source for brachytherapy coated with polydopamine prepared in Example 5 in the living tissue. These images are the CT images of XY plane, XZ plane, and YZ plane before and after the forced movement.

As shown in Table 2 and FIGS. 10 and 11, when the sealed source not-coated with polydopamine prepared in Comparative Example 1 was forced to move, it migrated 3.37 mm at farthest in the living tissue, while when the sealed source coated with polydopamine prepared in Example 5 was forced to move, it migrated 1.13 mm at farthest in the living tissue.

Therefore, the implantable medical appliance of the present invention displays the significantly reduced migration in the living tissue, so that it can be effectively used for the preparation of those implantable medical appliance with means of migration prevention after in vivo implantation.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. An implantable medical appliance characterized by the means equipped on the surface of the same to prevent intratissue migration.

2. The implantable medical appliance according to claim 1, wherein the surface of the implantable medical appliance is metal material.

3. The implantable medical appliance according to claim 2, wherein the metal material is one or more metals selected from the group consisting of titanium, stainless steel, iron, gold, silver, platinum, iridium, nickel, aluminium, tantalum, cobalt, chrome, and copper or an alloy composed of at least one of those metals selected from the same.

4. The implantable medical appliance according to claim 1, wherein the implantable medical appliance is selected from the group consisting of a sealed source for brachytherapy, a fiducial marker, a clip for surgical operation, and a transponder for the generation of RF (radio frequency).

5. The implantable medical appliance according to claim 4, wherein the sealed source for brachytherapy is selected from the group consisting of I-125, Pd-103, Ir-192, Au-198, Yb-169, Cs-131, Cs-137, and Co-60.

6. The implantable medical appliance according to claim 1, wherein the fiducial marker is characteristically the radiopaque material

7. The implantable medical appliance according to claim 1, wherein the means of migration prevention is the biocompatible polymer coated at least on a part of the implantable medical appliance that is also characterized by being increased in volume by absorbing body fluid.

8. The implantable medical appliance according to claim 7, wherein the biocompatible polymer is one or more compounds selected from the group consisting of chitosan, starch, guargum, gelatin, collagen, polylactide (PLA), polyglycolide (PGA), poly(lactic-co-glycolic acid) (PLGA), polyester, polyorthoester, polyanhydride, polyamino acid, polyhydroxybutyric acid, polycaprolactone, polyalkylcarbonate, and ethyl cellulose.

9. The implantable medical appliance according to claim 7, wherein the biocompatible polymer is characteristically biodegraded at least 60 days after in vivo implantation.

10. The implantable medical appliance according to claim 1, wherein the means of migration prevention is characterized by coating at least a part of the implantable medical appliance with the biocompatible adhesive before in vivo implantation of the medical appliance.

11. The implantable medical appliance according to claim 1, wherein the means of migration prevention is characterized by coating at least a part of the implantable medical appliance with the biocompatible adhesive after in vivo implantation of the medical appliance by injecting the biocompatible adhesive.

12. The implantable medical appliance according to claim 10, wherein the biocompatible adhesive is one or more compounds selected from the group consisting of polydopamine, cyanoacrylate, fibrin glue, protein glue, polyurethane, PEG containing sealant, and Azidobenzoic acid modified chitosan(Az-chitosan).

13. The implantable medical appliance according to claim 10, wherein the biocompatible adhesive is characteristically biodegraded at least 60 days after in vivo implantation.

14. The implantable medical appliance according to claim 11, wherein the biocompatible adhesive is one or more compounds selected from the group consisting of polydopamine, cyanoacrylate, fibrin glue, protein glue, polyurethane, PEG containing sealant, and Azidobenzoic acid modified chitosan(Az-chitosan).

15. The implantable medical appliance according to claim 11, wherein the biocompatible adhesive is characteristically biodegraded at least 60 days after in vivo implantation.

16. The implantable medical appliance according to claim 1, wherein the means of migration prevention is the foldable anchor equipped on the surface of the implantable medical appliance.

17. The implantable medical appliance according to claim 16, wherein the anchor is folded during the implantation but it will be unfolded after the implantation to be anchored in the surrounding tissues.
